(19) United States
(12) Patent Application Publication
(10) Pub. No.: US 2005/0147655 A1
(43) Pub. Date: Jul. 7, 2005

(54) Non-leaching antimicrobial glove

(76) Inventors: Alison S. Bagwell, Cumming, GA (US); David W. Johnson, Alpharetta, GA (US); David William Koenig, Menasha, WI (US); Martin S. Shamis, Alpharetta, GA (US); Wava M. Truscott, Roswell, GA (US)

Correspondence Address: KIMBERLY-CLARK WORLDWIDE, INC., 401 NORTH LAKE STREET, NEENAH, WI, 54956, US

(21) Appl. No.: 11/057,576

(22) Filed: Feb. 14, 2005

Related U.S. Application Data
(62) Division of application No. 10/355,579, filed on Jan. 31, 2003.

(51) Int. Cl. B32B 5/02; A61K 9/70

(52) U.S. Cl. 424/443; 442/123

(57) ABSTRACT

An elastomeric article having reducing microbe affinity and transmission is disclosed. The article has a coating of a hydrophobic, non-leaching antimicrobial polymer that is durably attached to an exterior surface, such that said antimicrobial polymer does not spontaneously migrate or is not removed from said exterior surface in the presence of aqueous substances, strong acids and bases, and organic solvents, and said antimicrobial polymer forms either a water-insoluable siloxane resin, or a covalently attached siloxane homopolymer, or a combination of both. The coated surface has a reduced affinity for aqueous-based substances and exhibits an enduring reduction in microbe affinity and transmission.

### RELATED APPLICATION

The present Application is a Divisional of U.S. patent application Ser. No. 10/355,579, filed Jan. 31, 2003, and claims priority thereto.

### BACKGROUND

Nosocomial, or hospital-acquired, infections occur in thousands of patients each year. Although use of aseptic techniques may reduce the incidence of these infections, a significant risk remains. In recent years, the need for improvement in the quality of patient care has received increasing attention, particularly infection control. A disposable glove that reduces the potential for transmission between inanimate objects and the patient, or the health care worker and the patient, i.e., contact transfer, may significantly reduce the likelihood of the patient contracting a hospital-acquired infection. This reduction in infection rates may reduce the amount of antibiotics used, therefore reducing the rate at which microbes become antimicrobial resistant. Additional benefits of reduced infection rates may include reduction in patient length of hospital stay, reduction in health care costs associated with hospital-acquired infections, and reduction in danger of infection to health care workers. As such, there is a need for a disposable glove that features a mechanism for reducing microbe affinity and transmission.

### SUMMARY OF THE INVENTION

The present invention relates to an elastomeric article, such as a glove, condom, a balloon, medical devices, or apparel that may require a mechanism for reducing an affinity for aqueous-based substances and exhibits a relatively prolonged duration or enduring reduction in microbe affinity and transmission. The elastomeric article has a substrate body with an exterior surface with at least a partial coating of a hydrophobic, non-leaching antimicrobial composition or polymer. As used herein, an “exterior surface,” refers to the surface of an article that will be remote or directed away from a user's skin or body. The antimicrobial polymer is adapted to disrupt either the cellular membrane structure or other bio-integrity of infectious organisms. The antimicrobial polymer that is at least partially covalently bonded to the exterior surface, which in-part contributes to a durable attachment of the antimicrobial molecules to the coated surface. The bulk or virtually all of the antimicrobial material should remain durably attached on the surface and retain its biocidal effectiveness for at least 48 hours from time of first exposure or use of the article by the user. The antimicrobial polymer can include an organosilane quaternary ammonium compound, such as 3-(trimethoxysilyl) propyldimethyloctadecyl ammonium chloride. The antimicrobial polymer may be a water-insoluble siloxane resin, a siloxane homopolymer, or a combination thereof. The article may include from about 0.05% to about 10% by mass of antimicrobial polymer. Desirably, the article has from about 2% to about 6% by mass of antimicrobial polymer. The antimicrobial polymer coating can be either continuous or discontinuous over the exterior surface.

The invention pertains to a glove made of an elastomeric material, such as either natural rubber latex or a synthetic polymer latex substrate, with an exterior surface having a coating of a hydrophobic, non-leaching antimicrobial polymer that is durably attached to said exterior surface. The antimicrobial polymer does not spontaneously migrate or is not removed from said exterior surface in the presence of aqueous substances, strong acids and bases, and organic solvents, and the antimicrobial polymer forms either a water-insoluable siloxane resin, or a covalently attached siloxane homopolymer, or a combination of both.

In another aspect, the invention relates to a method of making an elastomeric glove. The method includes: a) forming a glove from a natural or synthetic elastomeric material, with an exterior surface; b) applying to said exterior surface a hydrophobic, non-leaching antimicrobial composition that includes an organosilane quaternary ammonium compound, such that the antimicrobial composition at least partially hydrolyzes to form a water-insoluable siloxane resin, or polymerizes to form a covalently attached siloxane homopolymer, or a combination of both on the exterior surface. The antimicrobial composition is at least partially covalently attached to said exterior surface.

### BRIEF DESCRIPTION OF THE DRAWING

FIG. 1 depicts an elastomeric article, such as a glove, that may embody the present invention.

### DETAILED DESCRIPTION OF THE INVENTION

The present invention generally relates to an elastomeric article, for example, a glove, that reduces microbe affinity and transmission. The glove further has antimicrobial characteristics both during use and after disposal. As used herein, “antimicrobial” refers to the property of a compound, product, composition, or article that enables it to prevent or reduce the growth, spread, propagation, or other livelihood of a microbe. As used herein, “microbe” or “microbes” refers to any organism or combination of organisms likely to cause infection, such as bacteria, viruses, protozoa, yeasts, or molds.

The glove of the present invention includes a layer of a non-leaching antimicrobial polymer that is durably bonded to the exterior surface of glove. As used herein, “non-leaching” refers to the property of a material that renders it unlikely to or incapable of spontaneously migrating or being removed from the surface to which the material is applied. The antimicrobial polymer layer is formed from an antimicrobial composition, as defined and described herein.

The antimicrobial composition generally includes a silane ammonium quaternary compound, or organosilane, in a suitable solvent. One example of a composition that is effective when externally bound to a glove is Microbeshield™, an organosilane commercially available from Aegis Environments in Midland, Mich. The Microbeshield™ product line includes various combinations of 3-(trimethoxysilyl) propyldimethyloctadecyl ammonium chloride in methanol. For example, according to product literature, AEM 5700 is 43% 3-(trimethoxysilyl) propyldimethyloctadecyl ammonium chloride in methanol (with small percentages of other inactives) and AEM 5772 is 72% 3-(trimethoxysilyl) propyldimethyloctadecyl ammonium chloride in methanol (with small percentages of other inactives). While the Microbeshield™ compositions are described in detail herein, the present invention contemplates use of other silane quaternary ammonium compounds to form other antimicrobial compositions, such as those described in U.S. Pat. No. 4,631,273 to Blehm et al., herein incorporated by reference in its entirety.

When applied to a glove and processed as described herein, the antimicrobial composition, such as a Microbeshield™ composition, may at least partially covalently bond to the surface of the glove, and may at least partially homopolymerize, forming a covalently bonded antimicrobial polymer layer on the exterior surface of the glove. Short range forces, such as Van der Waals forces, may also contribute to the durability of the polymer layer on the glove. The antimicrobial polymer layer may be continuous or discontinuous on the surface of the glove.

Use of a durably bonded antimicrobial polymer reduces both microbe affinity to the glove and viable microbe transfer from the glove. First, the hydrophobic nature of the polymer on the glove reduces the affinity to bodily fluids, and therefore, the organisms contained therein. Furthermore, the organosilane is an antimicrobial chemistry that reduces livelihood and propagation of organisms in contact with the glove, so that transfer of microbes is decreased both during and after glove use. While not wishing to be bound by any particular theory, it is believed that the chemical structure of the antimicrobial polymer of the present invention disrupts the membrane structure of the microbial cell, causing unlinking of the proton motive force and rupture of the cell membrane.

Another beneficial aspect of the glove of the present invention is that the antimicrobial polymer formed on the surface of the glove is non-leaching in the presence of aqueous substances, strong acids and bases, and organic solvents. Traditional agents leach from the surface of the article, such as the glove, and must be consumed by the microbe to be effective. When such traditional agents are used, the microbe is poisoned and destroyed only if the dosing is lethal. If the dosing is sublethal, the microbe may adapt and become resistant to the agent. As a result, hospitals are reluctant to introduce such agents into the sterile environment. Furthermore, because these antimicrobial agents are consumed in the process, the efficacy of the antimicrobial treatment decreases with use.

The antimicrobial polymer used with the present invention does not leach from the surface of the glove, nor is it consumed by the microbe. Rather, the antimicrobial polymer ruptures the membrane of microbes that are present on the glove surface. Because the antimicrobial polymer is bound to the surface of the glove, it is a more durable chemistry that will provide an antimicrobial benefit for a longer duration.

The present invention further includes a method of making an elastomeric article, for example a glove, having reduced microbe affinity and transmission. The method generally includes forming an article having a surface, contacting the surface with a composition including an antimicrobial silane quaternary ammonium compound, and drying the article such that the compound at least partially hydrolyzes to form a water-insoluble siloxane resin, and at least partially homopolymerizes, thereby forming an antimicrobial polymer layer on the surface. To better understand the present invention, the entirety of the process is described below.

An elastomeric article to be treated, for example, a glove, may be formed using a variety of processes, for example, dipping, spraying, tumbling, drying, and curing. An exemplary dipping process for forming a glove is described herein, though other processes may be employed to form various articles having different shapes and characteristics. For example, a condom may be formed in substantially the same manner, although some process conditions may differ from those used to form a glove. Although a batch process is described and shown herein, it should be understood that semi-batch and continuous processes may also be utilized with the present invention.

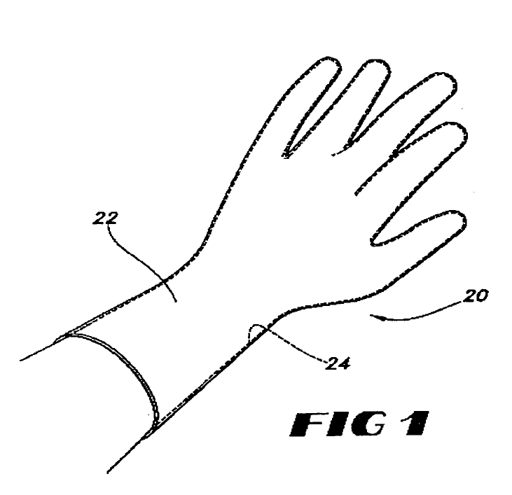

FIG. 1 illustrates a glove 20 that is formed on a hand-shaped mold, called a “former.” The former may be made from any suitable material, such as glass, metal, porcelain, or the like. The surface of the former defines at least a portion of the surface of the glove 20 to be manufactured. The glove 20 includes an exterior surface 22 and an interior surface 24. The interior surface 24 is generally the wearer-contacting surface.

The former is conveyed through a preheated oven to evaporate any water present. The former may then dipped into a bath typically containing a coagulant, a powder source, a surfactant, and water. The coagulant may contain calcium ions (from e.g., calcium nitrate) that enable a polymer latex to deposit onto the former. The powder may be calcium carbonate powder, which aids release of the completed glove from the former. The surfactant provides enhanced wetting to avoid forming a meniscus and trapping air between the form and deposited latex, particularly in the cuff area. However, any suitable coagulant composition may be used, including those described in U.S. Pat. No. 4,310,928 to Joung, incorporated herein in its entirety by reference. The residual heat evaporates the water in the coagulant mixture leaving, for example, calcium nitrate, calcium carbonate powder, and the surfactant on the surface of the former. Although a coagulant process is described herein, it should be understood that other processes may be used to form the article of the present invention that do not require a coagulant. For instance, in some embodiments, a solvent-based process may be used. The coated former is then dipped into a polymer bath, which is generally a natural rubber latex or a synthetic polymer latex. The polymer present in the bath includes an elastomeric material that forms the body of the glove. In some embodiments, the elastomeric material, or elastomer, includes natural rubber, which may be supplied as a compounded natural rubber latex. Thus, the bath may contain, for example, compounded natural rubber latex, stabilizers, antioxidants, curing activators, organic accelerators, vulcanizers, and the like. In other embodiments, the elastomeric material may be nitrile butadiene rubber, and in particular, carboxylated nitrile butadiene rubber. In other embodiments, the elastomeric material may be a styrene-ethylene-butylene-styrene block copolymer, styrene-isoprene-styrene block copolymer, styrene-butadiene-styrene block copolymer, styrene-isoprene block copolymer, styrene-butadiene block copolymer, synthetic isoprene, chloroprene rubber, polyvinyl chloride, silicone rubber, polyurethane, or a combination thereof.

The stabilizers may include phosphate-type surfactants. The antioxidants may be phenolic, for example, 2,2′-methylenebis (4-methyl-6-t-butylphenol). The curing activator may be zinc oxide. The organic accelerator may be dithiocarbamate. The vulcanizer may be sulfur or a sulfur-containing compound. To avoid crumb formation, the stabilizer, antioxidant, activator, accelerator, and vulcanizer may first be dispersed into water by using a ball mill and then combined with the polymer latex.

During the dipping process, the coagulant on the former causes some of the elastomer to become locally unstable and coagulate onto the surface of the former. The elastomer coalesces, capturing the particles present in the coagulant composition at the surface of the coagulating elastomer. The former is withdrawn from the bath and the coagulated layer is permitted to fully coalesce, thereby forming the glove. The former is dipped into one or more baths a sufficient number of times to attain the desired glove thickness. In some embodiments, the glove may have a thickness of from about 0.004 inches (0.102 mm) to about 0.012 inches (0.305 mm).

The former may then be dipped into a leaching tank in which hot water is circulated to remove the water-soluble components, such as residual calcium nitrates and proteins contained in the natural rubber latex and excess process chemicals from the synthetic polymer latex. This leaching process may generally continue for about 12 minutes at a water temperature of about 120° F. The glove is then dried on the former to solidify and stabilize the glove. It should be understood that various conditions, processes, and materials used to form the glove. Other layers may be formed by including additional dipping processes. Such layers may be used to incorporate additional features into the glove.

The glove is then sent to a curing station where the elastomer is vulcanized, typically in an oven. The curing station initially evaporates any remaining water in the coating on the former and then proceeds to a higher temperature vulcanization. The drying may occur at a temperature of from about 85° C. to about 95° C., and the vulcanizing may occur at a temperature of from about 110° C. to about 120° C. For example, the glove may be vulcanized in a single oven at a temperature of 115° C. for about 20 minutes. Alternatively, the oven may be divided into four different zones with a former being conveyed through zones of increasing temperature. For instance, the oven may have four zones with the first two zones being dedicated to drying and the second two zones being primarily for vulcanizing. Each of the zones may have a slightly higher temperature, for example, the first zone at about 80° C., the second zone at about 95° C., a third zone at about 105° C., and a final zone at about 115° C. The residence time of the former within each zone may be about ten minutes. The accelerator and vulcanizer contained in the latex coating on the former are used to crosslink the elastomer. The vulcanizer forms sulfur bridges between different elastomer segments and the accelerator is used to promote rapid sulfur bridge formation.

Upon being cured, the former may be transferred to a stripping station where the glove is removed from the former. The stripping station may involve automatic or manual removal of the glove from the former. For example, in one embodiment, the glove is manually removed and turned inside out as it is stripped from the former. By inverting the glove in this manner, the exterior of the glove on the former becomes the inside surface of the glove. It should be understood that any method of removing the glove from the former may be used, including a direct air removal process that does not result in inversion of the glove.

The solidified glove, or a plurality of solidified gloves, may then subjected to various post-formation processes, including application of one or more treatments to at least one surface of the glove. For instance, the glove may be halogenated to decrease tackiness of the interior surface. The halogenation (e.g., chlorination) may be performed in any suitable manner, including: (1) direct injection of chlorine gas into a water mixture, (2) mixing high density bleaching powder and aluminum chloride in water, (3) brine electrolysis to produce chlorinated water, and (4) acidified bleach. Examples of such methods are described in U.S. Pat. No. 3,411,982 to Kavalir; U.S. Pat. No. 3,740,262 to Agostinelli; U.S. Pat. No. 3,992,221 to Homsy, et al.; U.S. Pat. No. 4,597,108 to Momose; and U.S. Pat. No. 4,851,266 to Momose, U.S. Pat. No. 5,792,531 to Littleton, et al., which are each herein incorporated by reference in their entirety. In one embodiment, for example, chlorine gas is injected into a water stream and then fed into a chlorinator (a closed vessel) containing the glove. The concentration of chlorine may be altered to control the degree of chlorination. The chlorine concentration may typically be at least about 100 parts per million (ppm). In some embodiments, the chlorine concentration may be from about 200 ppm to about 3500 ppm. In other embodiments, the chlorine concentration may be from about 300 ppm to about 600 ppm. In yet other embodiments, the chlorine concentration may be about 400 ppm. The duration of the chlorination step may also be controlled to vary the degree of chlorination and may range, for example, from about 1 to about 10 minutes. In some embodiments, the duration of chlorination may be about 4 minutes.

While still within the chlorinator, the chlorinated glove or gloves may then be rinsed with tap water at about room temperature. This rinse cycle may be repeated as necessary. The gloves may then be tumbled to drain the excess water. A lubricant composition may then be added into the chlorinator, followed by a tumbling process that lasts for about five minutes. The lubricant forms a layer on at least a portion of the interior surface to further enhance donning of the glove. In one embodiment, this lubricant may contain a silicone or silicone-based component. As used herein, the term “silicone” generally refers to a broad family of synthetic polymers that have a repeating silicon-oxygen backbone, including, but not limited to, polydimethylsiloxane and polysiloxanes having hydrogen-bonding functional groups selected from the group consisting of amino, carboxyl, hydroxyl, ether, polyether, aldehyde, ketone, amide, ester, and thiol groups. In some embodiments, polydimethylsiloxane and/or modified polysiloxanes may be used as the silicone component in accordance with the present invention. For instance, some suitable modified polysiloxanes that may be used in the present invention include, but are not limited to, phenyl-modified polysiloxanes, vinyl-modified polysiloxanes, methyl-modified polysiloxanes, fluoro -modified polysiloxanes, alkyl-modified polysiloxanes, alkoxy-modified polysiloxanes, amino-modified polysiloxanes, and combinations thereof. Examples of commercially available silicones that may be used with the present invention include DC 365 available from Dow Coming Corporation (Midland, Mich.), and SM 2140 available from GE Silicones (Waterford, N.Y.). However, it should be understood that any silicone that provides a lubricating effect may be used to enhance the donning characteristics of the glove. The lubricant solution is then drained from the chlorinator and may be reused if desired. It should be understood that the lubricant composition may be applied at a later stage in the forming process, and may be applied using any technique, such as dipping, spraying, immersion, printing, tumbling, or the like.

The coated glove may then put into a tumbling apparatus or other dryer and dried for about 10 to about 60 minutes (e.g., 40 minutes) at from about 20° C. to about 80° C. (e.g., 40° C.). The glove may then be inverted to expose the exterior surface, which may then be dried for about 20 to about 100 minutes (e.g., 60 minutes) at from about 20° C. to about 80° C. (e.g., 40° C.).

After the various processes described above, the glove may be inverted (if needed) to expose the exterior surface of the elastomeric article, for example, the glove. Any treatment, or combination of treatments, may then be applied to the exterior surface of the glove. Individual gloves may be treated or a plurality of gloves may be treated simultaneously. Likewise, any treatment, or combination of treatments, may be applied to the interior surface of the glove. Any suitable treatment technique may be used, including for example, dipping, spraying, immersion, printing, tumbling, or the like.

In some embodiments, a treatment that reduces microbe affinity and viable transmission may be used. One such treatment that may be used is Microbeshield™, discussed above in detail. Microbeshield™ is available from Aegis Environments (Midland, Mich.) as various compositions of 3-(trimethoxysilyl) propyldimethyloctadecyl ammonium chloride in methanol. Two such compositions include AEM 5700 (43% total solids content) and AEM 5772 (72% total solids content).

To apply the composition to the gloves, a plurality of gloves may be placed in a closed vessel, where the gloves are immersed in an aqueous solution of the antimicrobial composition, for example, AEM 5700 or AEM 5772. In some embodiments, the antimicrobial composition may be added to water so that the resulting treatment includes about 0.05 mass % to about 10 mass % solids. In other embodiments, the antimicrobial composition may be added to water so that the resulting treatment includes from about 0.5 mass % to about 7 mass % solids. In other embodiments, the antimicrobial composition may be added to water so that the resulting treatment includes from about 2 mass % to about 6 mass % solids. In still another embodiment, the antimicrobial composition may be added to water so that the resulting treatment includes about 3 mass % solids. The gloves may be agitated if desired. The duration of the immersion may be controlled to vary the degree of treatment and may range, for example, from about 1 to about 10 minutes. For instance, the gloves may be immersed for about 6 minutes. The gloves may be immersed multiple times as needed to achieved the desired treatment level. For instance, the glove may undergo 2 immersion cycles.

The gloves may then be rinsed as needed to remove any excess antimicrobial composition. The gloves may be rinsed in tap water and/or deionized water as desired. After the gloves have been sufficiently rinsed, the excess water is extracted from the vessel and the gloves may be transferred to a tumbling apparatus or other dryer. The gloves may be dried for about 10 to about 60 minutes at from about 20° C. to about 80° C. For instance, the exterior surface of the gloves may be dried for about 40 minutes at a temperature of about 65° C. The gloves may then be inverted to expose the interior surface, which may then be dried for about 10 to about 60 minutes (e.g., 40 minutes) at from about 20° C. to about 80° C. For instance, the interior surface of the gloves may be dried for about 40 minutes at a temperature of about 40° C.

While not wishing to be bound by any particular theory, it is believed that during the immersion and drying process, the antimicrobial composition, in particular, the silane quaternary ammonium compound, at least partially hydrolyzes and at least partially polymerizes (i.e., homopolymerizes) to form at least two derivatives, namely, a highly-crosslinked, water insoluble siloxane resin, a covalently bonded homopolymer, or a combination of both (herein referred to as “antimicrobial polymer”), on the exterior surface of the glove. As used herein, “resin” refers to an organic polymeric liquid that becomes a solid when converted to its final state for use.

The antimicrobial polymer may be formed on the gloves to any extent suitable for a given application. The amount of polymer formed on the glove may be adjusted to obtain the desired reduction in microbe affinity, resistance to growth, and resistance to contact transfer, and such amount needed may vary depending on the microbes likely to be encountered and the application for which the article may be used. In some embodiments, the composition may be applied to the glove so that the resulting antimicrobial polymer is present in an amount of from about 0.05 mass % to about 10 mass % of the resulting glove. In other embodiments, the resulting antimicrobial polymer may be present in an amount of from about 1 mass % to about 7 mass % of the resulting glove. In yet other embodiments, the resulting antimicrobial polymer may be present in an amount of from about 2 mass % to about 5 mass % of the resulting glove.

If desired, the antimicrobial composition may be emulsified using an ether or a polyol prior to use, as is described in U.S. Pat. No. 6,113,815 to Elfersy et al. and U.S. Pat. No. 6,120,587 to Elfersy et al., respectively. However, contrary to the teachings of the Elfersy et al. patents, which require use of an ether or a polyol, respectively, to stabilize the composition, the present inventors discovered that it is not necessary to emulsify the antimicrobial composition in this manner prior to use. Rather, the present inventors have found that the antimicrobial composition need only be combined with water to provide a stable and efficacious aqueous treatment, as is demonstrated by the Examples herein. The present invention further contemplates a method for determining viable microbe transmission levels of an article. Any article may be evaluated, for example, a glove, catheter, swab, blotter paper, medical instruments, fabric, or the like. The method generally includes applying an inoculum including a microbe to a first surface, contacting a transfer substrate to the first surface, extracting the transferred inoculum from the transfer substrate, permitting the extracted inoculum to incubate, and quantifying the microbe level to determine a percent recovery. As used herein, “inoculum” refers to any material containing at least one microbe that may act as a source of infection in a host.

The method of the present invention may be used to measure viable contact transfer of various microbes, including, for example, Aspergillus niger (American Type Culture Collection (ATCC®) No. 16404), Candida albicans (ATCC® No. 10231), Hepatits A HM175/18f (ATCC® No. VR-1402), Herpes simplex virus 1 GHSV-UL46D (ATCC® No. VR-1545), Acinetobacter baumannii (ATCC® No. 15149), Clostridium difficile (ATCC® No. 43594), Enterobacter cloacae (ATCC® No. 29249), Enterococcus faecalis (ATCC® No. 51299), Enterococcus faecium (ATCC® NO. 700221), Enterococcus hirae (ATCC® No. 10541), Escherichia coli (ATCC® No. 13706), Escherichia coli (ATCC® No. 31705), Mycobacterium smegmatis (ATCC® No. 10143), Mycobacterium tuberculosis (ATCC® 27294), Pseudomonas aeruginosa (ATCC® No. 9027), Pseudomonas aeruginosa (ATCC® No. 27853), Staphylococcus aureus (ATCC®) No. 6538), Staphylococcus aureus (ATCC® No. 33592), Staphylococcus epidermidis (ATCC® No. 12228), and Staphylococcus epidermidis (ATCC® No. 51625).

After the desired microbe is selected, an inoculum is prepared by diluting a stock culture of the microbe. The culture may be diluted to any desired level using deionized water, and in some instances, may be diluted to an inoculum level of from about 1×10⁶ colony forming units (CFU)/ml to about 3×10⁶ CFU/ml.

Prior to performing the evaluation, a sterile buffer solution may be prepared for later use. The buffer solution may be replaced about every two months. In some instances, the buffer solution may be a sterile phosphate buffered water. One such buffer solution may be prepared as described in Example 2, and may have a final concentration of about 0.3 mM.

The desired inoculum may then be placed aseptically onto a first surface. Any quantity of the desired inoculum may be used, and in some embodiments, a quantity of about 1 ml is applied to the first surface. Furthermore, the inoculum may be applied to the first surface over any desired area. In some instances, the inoculum may be applied over an area of about 7 inches (178 mm) by 7 inches (178 mm). The first surface may be made of any material capable of being sterilized. In some embodiments, the first surface may be made of stainless steel, glass, porcelain, a ceramic, synthetic or natural skin, such as pig skin, or the like.

The inoculum may then be permitted to remain on the first surface for a relatively short amount of time, for example, about 2 minutes before the article to be evaluated, i.e., the transfer substrate, is brought into contact with the first surface.

The transfer substrate may be any article, and in some instances, is a surgical or examination glove. The transfer substrate, for example, the glove, should be handled aseptically. Where the transfer substrate is a glove, a glove may be placed on the left and right hands of the experimenter. One glove may then be brought into contact with the inoculated first surface, ensuring that the contact is firm and direct to minimize error. The test glove may then be immediately removed using the other hand and placed into a flask containing a desired amount of sterile buffered water (prepared above) to extract the transferred microbes. In some instances, the glove may be placed into a flask containing about 100 ml of sterile buffered water and tested within a specified amount of time. Alternatively, the glove may be placed into a flask containing a suitable amount of Letheen Agar Base (available from Alpha Biosciences, Inc. of Baltimore, Md.) to neutralize the antimicrobial treatment for later evaluation. The flask containing the glove may then be placed on a reciprocating shaker and agitated at a rate of from about 190 cycles/min. to about 200 cycles/min. The flask may be shaken for any desired time, and in some instances is shaken for about 2 minutes.

The glove may then be removed from the flask, and the solution diluted as desired. A desired amount of the solution may then be placed on at least one agar sample plate. In some instances, about 0.1 ml of the solution may be placed on each sample plate.

The solution on the sample plates may then be incubated for a desired amount of time to permit the microbes to propagate. In some instances, the solution may incubate for at least about 48 hours. The incubation may take place at any optimal temperature to permit microbe growth, and in some instances may take place at from about 33° C. to about 37° C. In some instances, the incubation may take place at about 35° C.

After incubation is complete, the microbes present are counted and the results are reported as CFU/ml. The percent recovery may then be calculated by dividing the extracted microbes in CFU/ml by the number present in the inoculum in (CFU/ml), and multiplying the value by 100.

The various aspects of the present invention may be better understood with reference to the following examples.

### EXAMPLE 1

The ability to modify the hydrophobicity of the exterior surface of a glove was demonstrated. A powder-free natural rubber examination glove commercially available from Safeskin Corporation under the trade name PFE Powder-Free Exam was immersed in an aqueous solution of about 5% AEM 5700 (43% total solid content) for about 1 minute while the solution was being stirred. The glove was then placed in an oven at about 80° C. for about 20 minutes. The glove was then rinsed twice in deionized water having a temperature of about 25° C. The glove was then tumble dried for about 20 minutes at about 55° C. The glove was then re-inverted to expose the interior surface and tumble dried for about 20 minutes at about 55° C. The glove was then inverted to expose the outside surface.

The static contact angle of deionized water on the exterior surface of the glove was then measured using a Rame-Hart Inc. NRL C.A. goniometer equipped with a Hitachi CCD camera. The water was obtained from a Gradient A10 MilliQ water purification system.

The contact angle on the exterior surface of the treated glove was measured to be 103°. The same instrument was used to measure the contact angle of deionized water on a control (untreated) glove. The contact angle was measured to be 0°. The hydrophobicity of the glove was thus increased by the presence of the antimicrobial polymer on the surface of the glove. Thus, the treated glove exhibited a decreased affinity to aqueous substances, thereby reflecting a likely reduced affinity to bodily fluids and the microbes contained therein.

### EXAMPLE 2

The potential for microbe transmission via untreated (non-antimicrobial) gloves was demonstrated. Using the following contact transfer test method contemplated by the present invention, powder free natural rubber examination gloves commercially available from Safeskin Corporation under the trade name PFE Powder-Free Exam were evaluated.

### Buffer Solution Preparation

A stock buffer solution of 0.25M KH₂PO₄ was prepared by adding about 34 g of potassium dihydrogen phosphate (KH₂PO₄) to about 500 ml of deionized water. The pH was then adjusted to about 7.2 with a dilute solution of NaOH. The solution was then diluted to about 1000 ml by adding deionized water. The diluted solution was then transferred to a flask and stored at about 4° C. A working stock buffer solution of about 0.3 mM KH₂PO₄ was then prepared by transferring about 1 ml of stock buffer solution with a sterile pipette to a flask containing about 800 ml of deionized water. The solution was mixed and dispensed into 100 ml volumes in 250 ml Erlenmeyer flasks. The flasks were capped with a sponge and foil, and sterilized at about 121° C. and about 18-22 psig for about 20 minutes using a liquid sterilization cycle.

### Testing Protocol

A stock culture of S. aureus was diluted three times using 1:9 serial dilutions to reach various final desired test inoculum level of from about 1×10⁶ to about 3×10⁶ CFU/ml. Dilution blanks having a volume of 9 ml were then prepared using sterile deionized water.

One ml of each of the prepared inocula was aseptically spread onto a stainless steel surface (back side of a stainless steel instrument tray) over an area of about 7 in. (178 mm) by about 7 in. (178 mm) using a sterile cotton tipped applicator. Each inoculum was allowed to remain on the surface for about 2 minutes (the surface was re-wet with deionized water before contacting with test gloves).

A test glove was aseptically placed on the left and right hands. The glove on the right hand was then contacted firmly to the surface, with all fingers and the thumb touching the inoculated area. Using the left gloved hand, the right hand glove was removed immediately and aseptically placed into a 250 ml Erlenmeyer flask containing about 100 ml of the sterile phosphate buffered water prepared above. The flask was then placed on a reciprocating shaker and agitated at about 195 cycles/min. for about 2 minutes.

Serial dilutions of 1:9 were then performed on the flask solution using the 9 ml sterile deionized water blanks. The diluted flask solution was then placed on agar sample plates (about 0.1 ml per plate) for incubation. The sample plates were incubated for about 48 hours at about 35° C. to permit bacterial growth. After incubation, the microbe presence on each plate was counted to determine the percent recovery. The results were reported as CFU/ml to determine percent (%) recovery, and are listed in Table 1.

| Sample | Inoculum Level | CFU Recovered | % Recovery |
|---|---|---|---|
| 1 | 3.3 × 10⁶ | 1.7 × 10⁵ | 5.1 |
| 2 | 3.3 × 10⁶ | 3.4 × 10⁴ | 1.0 |
| 3 | 3.3 × 10⁶ | 1.5 × 10⁵ | 4.5 |
| 4 | 3.3 × 10⁶ | 2.9 × 10⁴ | 0.9 |
| 5 | 3.3 × 10⁶ | 2.2 × 10⁵ | 6.7 |
| 6 | 1.2 × 10⁶ | 1.2 × 10⁵ | 10.0 |
| 7 | 1.2 × 10⁶ | 1.4 × 10⁵ | 11.6 |
| 8 | 1.2 × 10⁶ | 8.7 × 10³ | 0.7 |
| 9 | 1.2 × 10⁶ | 1.9 × 10⁵ | 15.8 |
| 10 | 1.2 × 10⁶ | 1.7 × 10⁵ | 14.1 |

For each sample evaluated, some amount of S. aureus was transferred from the stainless steel surface to the glove. Thus, untreated natural rubber examination gloves are a possible source for contact transfer of viable S. aureus. If other microbes behave similarly to S. aureus, natural rubber examination gloves are a possible source for contact transfer for various microbes.

### EXAMPLES 3-5

The non-leaching nature of the antimicrobial polymer was demonstrated. Furthermore, reduction in microbe transmission of the glove of the present invention was demonstrated. To prepare the samples used in Examples 3-5, PFE Powder-Free Exam gloves available from Safeskin Corporation were first rinsed two times in 1500 L of tap water for about 6 minutes per rinse (with the exterior surface of the gloves exposed). The gloves were then immersed into about 750 L of various aqueous solutions of AEM 5700 (43% total solids content) in water, to which a small amount of a surfactant was added (less than 0.1 mass % of the solution mass). The total solids content of each solution evaluated is presented below. The gloves were then rinsed two times in about 1500 L of tap water for about 6 minutes per rinse, and then rinsed two times in about 1500 L deionized water for about 6 minutes per rinse. The gloves were then dried in an oven at about 65° C. for about 40 minutes, inverted to expose the interior surface, and dried at about 55° C. for about 40 minutes. Various tests were performed as described below.

### EXAMPLE 3

The non-leaching nature of the antimicrobial polymer on a glove was demonstrated using zone of inhibition testing according to Section 12 of ASTM E2149-01 entitled “Standard Test Method for Determining the Antimicrobial Activity of Immobilized Antimicrobial Agents Under Dynamic Contact Conditions”. The zone of inhibition is presented as a distance in millimeters from the source of an antimicrobial agent in which the antimicrobial is effective. The results are summarized below.

| | | | | | Zone of |
|---|---|---|---|---|---|
| | Number of | | AEM 5700 | | inhibition |
| Test | gloves | Water (g) | (%) | Solids (%) | (mm) |
| 1 | 130 | 19000 | 5.0 | 4.70 | 0 |
| 2 | 130 | 19000 | 4.5 | 4.25 | 0 |
| 3 | 130 | 19000 | 4.0 | 3.80 | 0 |
| 4 | 130 | 19000 | 3.5 | 3.34 | 0 |
| 5 | 130 | 19000 | 3.0 | 2.89 | 0 |
| 6 | 130 | 19000 | 2.5 | 2.42 | 0 |
| 7 | 130 | 19000 | 2.0 | 1.95 | 0 |
| 8 | 260 | 19000 | 1.0 | 0.99 | 0 |

At each concentration evaluated, there was no zone of inhibition. Thus, the results demonstrate that the antimicrobial polymer formed on the exterior of the glove is non-leaching.

### EXAMPLE 4

The contact transfer test method described in Example 2 was then used to measure the percent recovery of the S. aureus inoculum. The results are summarized below.

| | Number of | | AEM 5700 | Solids | Percent recovery |
|---|---|---|---|---|---|
| Test | gloves | Water (g) | (%) | (%) | (CFU/ml) |
| 1 | 130 | 19000 | 5.0 | 4.70 | 0 |
| 2 | 130 | 19000 | 4.5 | 4.25 | 0 |
| 3 | 130 | 19000 | 4.0 | 3.80 | 0 |
| 4 | 130 | 19000 | 3.5 | 3.34 | 16.7 |
| 5 | 130 | 19000 | 3.0 | 2.89 | 77.5 |
| 6 | 130 | 19000 | 2.5 | 2.42 | No reduction |
| 7 | 130 | 19000 | 2.0 | 1.95 | No reduction |
| 8 | 260 | 19000 | 1.0 | 0.99 | No reduction |

The results demonstrate that at total solids content levels of about 3.34%, there is a significant reduction in contact transfer of viable S. aureus via the tested gloves, and at total solids content levels of about 3.80, there is no contact transfer via the tested gloves. It should be understood that the total solids content level may be modified to obtain the necessary resistance to contact transfer for various microbes and for various applications.

### EXAMPLE 5

The resistance of the gloves of the present invention to the growth of microbes under dynamic contact conditions was evaluated using ASTM E2149-01 entitled “Standard Test Method for Determining the Antimicrobial Activity of Immobilized Antimicrobial Agents Under Dynamic Contact Conditions”. The percent reduction was determined after about 3 minutes and after about 5 minutes. The results are summarized below.

| | | | AEM | | % | % |
|---|---|---|---|---|---|---|
| | Number of | | 5700 | Solids | Reduction | Reduction |
| Test | gloves | Water (g) | (%) | (%) | (3 min.) | (5 min.) |
| 1 | 130 | 19000 | 5.0 | 4.70 | 99.7 | 99.9 |
| 2 | 130 | 19000 | 4.5 | 4.25 | 99.9 | 99.9 |
| 3 | 130 | 19000 | 4.0 | 3.80 | 98.0 | 99.9 |
| 4 | 130 | 19000 | 3.5 | 3.34 | 98.3 | 99.9 |
| 5 | 130 | 19000 | 3.0 | 2.89 | 98.5 | 99.9 |
| 6 | 130 | 19000 | 2.5 | 2.42 | 22.2 | 33.3 |
| 7 | 130 | 19000 | 2.0 | 1.95 | 11.1 | 38.9 |
| 8 | 260 | 19000 | 1.0 | 0.99 | 38.9 | 38.9 |

At all total solids content levels evaluated, there was some reduction in S. aureus presence after 3 minutes and after 5 minutes. At or above a total solids content level of about 2.89%, there was a significant reduction in microbe presence, indicating that at such levels, the gloves of the present invention are highly resistant to microbe growth. It should be understood that the total solids content level may be modified to obtain the necessary resistance to microbe growth for various microbes and for various applications.

In summary, the glove of the present invention exhibits an increased hydrophobicity due to the presence of an antimicrobial polymer layer formed on the exterior of the glove. The increase in hydrophobicity decreases the affinity of bodily fluids and microbes to the glove. The antimicrobial polymer has further been demonstrated as non-leaching and effective at reducing microbe presence. The method of determining contact transfer of a microbe has been shown to produce results that are consistent with other test methods, demonstrating that it is an effective means of determining both the potential for transfer on an untreated article and the efficacy of a treated article.

The invention may be embodied in other specific forms without departing from the scope and spirit of the inventive characteristics thereof. The present embodiments therefore are to be considered in all respects as illustrative and not restrictive, the scope of the invention being indicated by the appended claims rather than by the foregoing description, and all changes which come within the meaning and range of equivalency of the claims are therefore intended to be embraced therein.

1. An elastomeric article comprising: a substrate body with an exterior surface having a coating of a hydrophobic, non-leaching antimicrobial polymer that is attached to said exterior surface, said antimicrobial polymer is adapted to disrupt either cellular membrane structure or other bio-integrity of infectious organisms, and said article exhibits a reduced affinity for aqueous-based substances and a reduced microbe affinity and transmission rate.

2. The elastomeric article according to claim 1, where antimicrobial polymer that is at least partially covalently bonded to said exterior surface.

3. The elastomeric article according to claim 1, where said article exhibits an enduring reduction in microbe affinity and transmission rate.

4. The elastomeric article according to claim 1, wherein said antimicrobial polymer is formed from an organosilane quaternary ammonium compound.

5. The elastomeric article according to claim 4, wherein said compound includes 3-(trimethylsilyl)-propyldimethyloctadecyl ammonium chloride.

6. The elastomeric article according to claim 1, wherein said antimicrobial polymer is selected from the group consisting of a water-insoluable siloxane resin, a siloxane homopolymer, and a combination thereof.

7. The elastomeric article according to claim 1, wherein said article comprises from about 0.05% to about 10% by mass of antimicrobial polymer.

8. The elastomeric article according to claim 7, wherein said article comprises from about 2% to about 6% by mass of antimicrobial polymer.

9. The elastomeric article according to claim 1, wherein said antimicrobial polymer coating is either continuous or discontinuous over said exterior surface.

10. The elastomeric article according to claim 1, wherein said article is a glove formed from either natural rubber latex or a synthetic polymer latex.

11. A glove comprising: an elastomeric material substrate, with an exterior surface having a coating of a hydrophobic, non-leaching antimicrobial polymer that is durably attached to said exterior surface, such that said antimicrobial polymer does not spontaneously migrate or is not removed from said exterior surface in the presence of aqueous substances, strong acids and bases, and organic solvents, and said antimicrobial polymer forms either a water-insoluable siloxane resin, or a covalently attached siloxane homopolymer, or a combination of both.

12. A method of making an elastomeric glove, the method comprises: a) forming a glove from a natural or synthetic elastomeric material, with an exterior surface; b) applying to said exterior surface a hydrophobic, non-leaching antimicrobial composition that includes an organosilane quaternary ammonium compound, such that said antimicrobial composition at least partially hydrolyzes to form a water-insoluable siloxane resin, or polymerizes to form a covalently attached siloxane homopolymer, or a combination of both on said exterior surface.

13. The method according to claim 12, wherein said antimicrobial composition is at least partially covalently attached to said exterior surface.
